(19) United States
(12) Patent Application Publication
(10) Pub. No.: US 2014/0271610 A1
(43) Pub. Date: Sep. 18, 2014

(54) COLLAGEN AND ALGINATE-BASED FORMULATIONS FOR HEMOSTASIS

(71) Applicant: ORTHOVITA, INC., Malvern, PA (US)

(72) Inventor: James San Antonio, Media, PA (US)

(73) Assignee: ORTHOVITA, INC., Malvern, PA (US)

(21) Appl. No.: 13/796,068

(22) Filed: Mar. 12, 2013

(51) Int. Cl. A61L 26/00

(52) U.S. Cl. CPC A61L 26/0052
USPC 424/94.67; 514/13.7; 424/683; 514/9.3

(57) ABSTRACT

The present invention relates to composition(s) comprising collagen, calcium ions and alginate, wherein the composition polymerizes in situ when contacted with blood. The composition may further comprise an erythrocyte aggregation agent and/or a platelet activation agent. The composition may be formulated into two separate formulations. The first formulation comprises collagen in a calcium-containing solution and a second formulation comprises an aqueous alginate solution. The two formulations can be mixed prior to the contact with blood or mixed coincident with the contact with blood.

### BACKGROUND OF THE INVENTION

There are many different hemostat products available in the market to enhance blood clotting reactions. Many of the products use thrombin to produce a hemostat.

The concept of using alginate as a bandage or a wound dressing is discussed in the art. For example, U.S. Pat. No. 7,279,177 describes alginate as one of many polymers that might be used in a cellulose fabric hemostatic wound dressing. U.S. Pat. No. 6,911,437 describes the use of alginate as one of various woven wound covers. Some of the commercially available products include AlgiSite M Calcium Alginate Dressing, sold by Smith and Nephew, Inc., Fibracol Collagen-Alginate Wound Dressing by Moore Medical, Curasorb Calcium Alginate Dressing by Kendall, and Alginate Hemostatic Pad by Non-Woven Fabric Co., Ltd.

These products, however, are limited to the use of pre-polymerized and processed alginate. Accordingly, there is a need to a composition which promotes hemostasis and promotes the clotting cascade within the patient's blood and/or tissue at the surgical site via alginate polymerization in situ.

### BRIEF SUMMARY OF THE INVENTION

The present invention is directed to a composition comprising collagen, calcium ions and alginate to promote hemostasis which can polymerize in situ at the surgical site. In one embodiment, the composition further comprises an erythrocyte aggregation agent and/or a platelet activation agent. In another embodiment, the composition further comprises a collagen bridging protein. In some embodiments, the collagen may be biotinylated. In one embodiment, the collagen is crosslinked before delivery. In another embodiment, the collagen is crosslinked during delivery. In one embodiment, the composition of the present invention does not contain thrombin and/or human plasma.

In various embodiments, the first formulation comprises collagen in a calcium-containing solution and a second formulation comprises an aqueous alginate solution, wherein the alginate is non-polymerized. The two formulations can be mixed prior to the contact with blood or mixed coincident with the contact with blood to promote rapid gelation and/or polymerization of the composition. The collagen in the first formulation is preferably microfibrillar collagen.

In some embodiments, hemostasis is promoted via high affinity covalent and/or non-covalent interactions between collagen, alginate and/or other components, such as an erythrocyte aggregation agent and/or a platelet activation agent to promote rapid gelation of the composition when in contact with blood and activation of the hemostasis cascade.

Methods of making these compositions and methods of using these compositions in hemostasis applications are also part of the invention. In one embodiment, a method of making a hemostat composition comprises mixing collagen, calcium ions and alginate to form a mixture; and delivering the mixture to a site of application, e.g., surgical (bleeding) site. In another embodiment, the method further comprises mixing the mixture comprising collagen, calcium ions and alginate with blood prior to delivering the mixture to a site of application. In yet another embodiment, the method comprises providing a flowable composition comprising collagen and calcium ions in a first container; providing a flowable composition comprising alginate in a second container; providing a device which would simultaneously mix the flowable compositions of the first and second containers and inject the composition to a site of application by the action of applied injection force.

### BRIEF DESCRIPTION OF THE DRAWINGS

FIG. 1 includes pictorial depictions of microfibrillar collagen, fibrin, non-polymerized alginate, polymerized alginate, red blood cells and platelets, a calcium ion and a sodium ion, which are also depicted in FIGS. 2 to 4.

FIG. 2 is a schematic of one embodiment of the present invention illustrating a highly magnified view of a polymerized alginate-microfibrillar collagen gel in which blood, clots (containing fibrin, red blood cells and platelets) have formed.

FIG. 3 is a schematic of one embodiment of the present invention wherein the first formulation comprising microfibrillar collagen in a calcium-containing solution and a second formulation comprising alginate are mixed first via a mixer, and then the mixture is further mixed with blood at the surgical (bleeding) site.

FIG. 4 is a schematic of yet another embodiment of the present invention wherein one or more mechanical mixing devices are used, to simultaneously mix the patient's blood with the first formulation comprising collagen in a calcium-containing solution and the second formulation comprising alginate prior to application to the surgical (bleeding site.

### DETAILED DESCRIPTION

The present invention will be described in more detail below.

All percentages and ratios used herein are by weight of the total composition and all measurements made are at about room temperature and normal pressure unless otherwise designated. “Room temperature” as defined herein means a temperature ranging between about 22° C. and about 26° C. All temperatures are in degrees Celsius unless specified otherwise.

The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise.

As used herein, “consisting essentially of” means that the invention may include ingredients in addition to those recited in the claim, but only if the additional ingredients do not materially alter the basic and novel characteristics of the claimed invention. Preferably, such additional ingredients will not be present at all or only in trace amounts. However, it may be possible to include up to about 10% by weight of materials that could materially alter the basic and novel characteristics of the invention as long as the utility of the compounds (as opposed to the degree of utility) is maintained.

All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

It should be further understood that a description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.3, 3, 4, 5, 5.7 and 6. This applies regardless of the breadth of the range.

Note that while the specification and claims may refer to a final product containing a certain material or a certain amount/concentration, it may be difficult to tell from the product that any particular recitation is satisfied. Such a recitation may be satisfied, however, if the materials used prior to final production, for example, meet that recitation. Indeed, as to any property or characteristic of a final product which cannot be ascertained from the final product directly, it is sufficient if that property resides in the components recited just prior to production steps used to make the composition.

Collagen

Collagen, preferably biocompatible collagen, is present in the composition in an amount sufficient to thicken the composition and augment its cohesive properties. The collagen may be a telopeptide collagen or telopeptide-containing collagen (e.g., native, native-type, or near-native collagen with or without enzymatic processing to remove N- and C-terminal telopeptides). In addition to thickening the composition, the collagen acts as a macromolecular lattice or scaffold. This feature gives more strength and durability to the resulting composition/clot. In addition, the collagen may be able to enhance gelation and set in a hemostat composition. In some embodiments, the collagen of the invention is prepared as described in U.S. Pat. Nos. 6,096,309 and 6,280,727 and U.S. Patent Publication Application No. 2011/0243913, which are incorporated by reference in their entirety.

One form of collagen that is employed may be described as at least “near native” or “native-type” in its structural characteristics. In various embodiments, the collagen may be characterized as resulting in insoluble fibers at a pH above 5; unless crosslinked or as part of a complex composition (e.g., bone). In some embodiments, the collagen will be predominantly microfibrillar, generally consisting of a minor amount by weight of fibers with diameters greater than 30 nm, preferably from about 1 wt % to about 30 wt %. In preferred embodiments, the collagen is microfibrillar type I collagen. Other forms of collagen which are employed may include microfibrillar collagen mixed with fibrillar collagen at various ratios, denatured collagen, or gelatin, or a mixture of microfibrillar collagen, fibrillar collagen and/or gelatin in varying proportions. Although collagen can take many forms: denatured and sometimes partially fragmented as in gelatin; monomeric with a native triple helical conformation as in procollagen; polymerised into a five-mer aggregate or higher molecular weight aggregates ≦30 nm in diameter as in microfibrillar collagen; or polymerized, into higher-ordered cable-like fibrils as in fibrillar collagen, in this invention a “collagen molecule” may be taken to describe any of these entities or molecular forms of collagen.

In preferred embodiments, the collagen is microfibrillar type I collagen. Microfibrillar collagen may have several advantages in the present invention applications. First, microfibrillar collagen has been shown to have strong platelet activating activity owing to its ability, via the presence of glycine-proline-hydroxyproline repeats and integrin binding sites in its triple helical domain, to ligate and activate platelet GPVI and α2β1 integrin receptors. Second, microfibrillar collagen assembles into collagen fibrils which provide a rigid, settable substrate and mesh-like network to support platelet adhesion and clot stabilization.

In other embodiments, the collagen may comprise microfibrillar or fibrillar collagen mixed with various concentrations of denatured collagen, or gelatin. In yet further embodiments of the present invention, the collagen may be comprised entirely of any concentration of gelatin.

In various embodiments, the collagen molecules described herein are covalently modified to possess sulfhydryl groups. In certain embodiments, the addition of sulfhydryl groups is helpful for promoting intermolecular and intramolecular associations. In its processed native form, type I collagen does not contain cysteine, an amino acid that has a sulfhydryl group side chain. Therefore, selected amino acid side chains on type I collagen can be covalently modified with cysteine, in various embodiments of the invention, using techniques known in the art for modifying collagen. (See 2011, He et al., Acta Biomater., 7:1084-1093; 2000, Myles et al., J Biomater Sci Polymer Ed, 44:69-86, which is incorporated herein by reference in its entirety).

In some embodiments, the collagen is present in a concentration ranging from about 0.5 mg/ml to about 15 mg/ml, preferably about 5 mg/ml to about 10 mg/ml in the composition. The amount of the collagen can be varied to provide compositions of differing viscosities and strengths, depending on the particular application.

In various embodiments, the collagen is in a physiologically acceptable liquid vehicle, such as an aqueous isotonic vehicle containing at least one salt, for example sodium chloride and calcium chloride. In an embodiment, sodium chloride is present, in a concentration ranging from about 15 mM to about 125 mM, preferably from about 15 mM to about 100 mM, and most preferably at about 75 mM in the composition.

Calcium Ions

Calcium ions are present in the composition in an amount sufficient to polymerize the alginate in the composition and to maintain collagen in the microfibrillar conformation. Non limiting examples of a source of calcium ions are calcium chloride, calcium lactate, calcium acetate, and mixtures thereof.

In a preferred embodiment, calcium ions are supplied separately from alginate to prevent premature polymerization of the composition. Calcium ions and alginate can be mixed shortly prior to or during delivery of the composition to a site, e.g., a surgical and/or any bleeding site.

In some embodiments, a concentration of calcium, ions present in the composition is greater than 7.5 mM. In other embodiments, a concentration of calcium ions present in the composition ranges from about 7.5 mM to about 75 mM, preferably from 10 to about 50 mM and most preferably at about mM.

Alginate

Alginate and derivatives thereof are present in the composition in an amount sufficient to induce rapid gelation of the composition when in contact with the collagen and calcium-containing solution and blood thereby concentrating and activating clotting cascade components and accelerating hemostasis. Preferably alginate used in accordance with the present invention is biocompatible, biodegradable, non-toxic, non-inflammatory and/or non-immunogenic. Alginate is provided in the formulation as a non-polymerized form, and is later polymerized in situ when combined/contacted with collagen, calcium ions and blood. In one embodiment, alginate includes alginate salts such as sodium or potassium alginate. In some embodiments, the alginate is present in an amount from about 0.1 mg/ml to about 50 mg/ml, preferably about 5 mg/ml to about 20 mg/ml in the composition

In various embodiments the alginate is in a physiologically acceptable liquid vehicle, such as an aqueous isotonic vehicle at about a physiologic salt concentration. The amount of the alginate can foe varied depending on the particular application.

Formulation/Kits/Method of Making

The compositions of the present invention can be formulated in various different configurations depending on the particular application. In one embodiment, a composition of the present invention is produced by mixing one or more formulations.

In various embodiments, the first formulation comprises collagen and calcium ions and a second formulation comprises alginate. In various other embodiments, the first formulation comprises collagen in a calcium-containing solution and a second formulation comprises an alginate solution. The composition in accordance with the present invention can be made by mixing the first and second formulations.

In another embodiment, one or more mechanical mixing devices are used, to simultaneously mix the patient's blood with the first formulation comprising collagen in a calcium-containing solution and the second formulation comprising alginate prior to application to site.

In yet another embodiment, the first formulation comprising collagen in a calcium-containing solution and a second formulation comprising alginate are mixed first, and then the mixture is further mixed with blood prior to application to site. In another embodiment, the first and second formulations are mixed first to form a mixture, and then the mixture is further mixed with blood at the site of application, e.g., a bleeding site.

In some embodiments, a kit is provided comprising a first container which comprises collagen and calcium ions and a second container which comprises alginate. In another embodiment, a kit comprises a first container comprising a flowable composition comprising collagen and calcium and a second container comprising a flowable composition comprising alginate. In one embodiment, the first container comprises a native or denatured collagen having a concentration ranging from about 1 mg/ml to about 30 mg/ml, preferably about 10 mg/ml to about 20 mg/ml. In another embodiment, the first container comprises a microfibrillar collagen having a concentration of about 1 mg/ml to about 30 mg/ml, preferably about 10 mg/ml to about 20 mg/ml. In some embodiments, the concentration of calcium ions present in the first container is greater than 15 mM. In other embodiments, a concentration of calcium ions present in the first container ranges from about 15 mM to about 150 mM, preferably from 20-100 mM and most preferably at about 40 mM.

In other embodiments where both calcium chloride and sodium chloride are present in the first container, the concentration of calcium chloride and the concentration of sodium chloride may not be both less than or equal to about 40 mM in order to maintain collagen in the microfibrillar conformation. In some embodiments, where both calcium chloride and sodium chloride are present in the composition, the concentrations of calcium chloride and sodium chloride are from about 20 mM to about 40 mM, and at about 150 mM, respectively; from about 20 mM to about 40 mM, and about 75 mM, respectively; from about 10 mM and from about 30 mM, and about 100 mM, respectively; at about 20 mM, and at about 300 mM, respectively; and from about 15 mM to about 15 mM, and from about 20 mM to about 500 mM, respectively.

In another embodiment, the second container comprises an aqueous solution comprising alginate having a concentration ranging from about 0.1 mg/ml to about 50 mg/ml, preferably about 5 mg/ml to about 20 mg/ml. In some embodiments, a two barrel syringe containing the flowable collagen- and calcium-containing composition and the flowable alginate composition in the respective barrel is employed to simultaneously mix and extrude the composition into the surgery site.

Erythrocyte Aggregating Promoter

An erythrocyte aggregation promoter is present in the composition in an amount sufficient to promote erythrocyte aggregation and/or to increase the hemostatic activity of the composition by targeting the aggregation of the predominant population of cells of the blood.

In an embodiment, the erythrocyte aggregating promoter is a charged, hydrophobic or neutral polymer having a molecular weight of greater than about 20 kD. Examples of such polymer having a molecular weight of greater than about 20 kD are, but not limited to, poly-L-lysine, poly-L-arginine, poly-L-ornithine, protamine, and similar cationic polymers; poly-L-glutamine, poly-L-aspartic acid, and similar anionic polymers and polyethylene glycol or dextran and similar hydrophobic or uncharged polymers. To avoid potentially disruptive effects of the polymers on the polymerization of alginate or on the solubility or polymerization of collagen, such as microfibrillar collagen, an anionic polymer or a weakly cationic polymer at physiologic pH may be used. The preferred pH range is at about 5 to about 8, preferably at about 6 to about 8.

In various embodiments of the kits in accordance with the present invention, the erythrocyte aggregation promoter may be included as an additional substance in either or both of the first container comprising the collagen solution and the second container comprising the alginate solution.

Platelet Activation Agent

A platelet activation agent is present in the composition in an amount sufficient to activate the platelets present in vivo and cause them to become sticky and adhere together to promote blood coagulation.

Examples of the platelet activating agents are, but not limited to adenosine diphosphate, 1-0-alkyl-2-acetyl-sn-glyceryl-3-phosphorylcholine, arachidonic acid, epinephrine, ristocetin, cycloheximide, N-monomethyl L-arginine, atrial naturetic factor (ANF), small nucleotides (including cAMP, cGMP, and ADP), prostaglandins, thromboxanes and analogs thereof, phorbols and phorbol esters, ethamsylate, and hemoglobin and mixtures thereof. Nonabsorbable powders such as talc, and denatured or surface-absorbed proteins can also activate platelets. In various embodiments of the kits in accordance with the present invention, the platelet activation agent may be included as an additional substance in either or both of the first container comprising the collagen solution and the second container comprising the alginate solution.

Crosslinkable Collagen and Crosslinking Agents

The collagen molecule preferably comprises at least one crosslinkable moiety that is able to form a bond, directly or indirectly, with another crosslinkable moiety on another collagen molecule. Any crosslinkable moieties known in the art may be used. By way of non-limiting examples, the collagen molecules can be crosslinked by covalent interactions, by non-covalent interactions, by thermally reversible interactions, by ionic interactions, or by combinations thereof. These moieties can be crosslinked by physical, chemical, thermal, or photoinitiation (e.g., visible and/or UV) means, or by any combination thereof.

In some embodiments, a transglutaminase is used to crosslink collagen molecules. Transglutaminases are known to catalyze the formation of covalent bonds between a free amine group on protein-bound lysines and the gamma-carboxamide group of protein-bound glutamines. Bonds formed by transglutaminase are resistant to proteolytic degradation. Non-limiting examples of transglutaminases useful in the compositions and methods of the invention include Factor XIII, a blood clotting cascade component, and Streptomyces mobaraensis transglutaminase (e.g., Activa TG™), and various forms of recombinant transglutaminases. In other embodiments, genipin or glutaraldehyde are used, alone or in combination with other crosslinkers, to crosslink collagen molecules in the compositions and methods of the invention.

In some embodiments, crosslinking of collagen may be carried out before use of the composition for hemostasis; while in other embodiments, crosslinking may foe carried out at the time of application to the patient. In various embodiments of the kits in accordance with the present invention, the crosslinking agent is added to the second, container comprising the alginate solution.

Biotinylated Collagen

In some embodiments, an avidin-biotin interaction may be employed to connect collagen molecules to one another. In one embodiment, the collagen molecules are biotinylated and the biotinylated collagen molecules are then crosslinked using a biotin-binding protein, such as avidin. Biotin can be attached to collagen by a variety of methods known in the art, including, by way of non-limiting example, the method reported by Lee at al. (2006, Mol Biol Cell 17: 4812-4826). Biotin-binding molecules useful in the compositions and methods of the invention include, by way of non-limiting examples, avidin, streptavidin, tamavidin, NeutrAvidin™ and CaptAvidin™.

In various embodiments of the kits in accordance with the present invention, the second container comprises avidin alginate mixture. Upon mixing the first container comprising collagen and calcium ions and the second container comprising avidin alginate mixture in accordance with one embodiment, and extrusion into the surgery site, the collagen populations would rapidly form a high affinity intermolecular scaffold with hemostatic properties, interspersed among the alginate gel.

Collagen Bridging Molecules

In various embodiments, the compositions of the invention further include a collagen bridging molecule. A collagen bridging molecule is any molecule that, binds to a collagen monomer or fibril, and which is bi- or multivalent for collagen binding. As such, a collagen bridging molecule, when mixed with collagen, can bind to more than one collagen molecule and form a relatively stable, high or low affinity, non-covalent interaction. A collagen bridging molecule can also help form a more cohesive intermolecular scaffold between collagen molecules. Collagen bridging molecules that exhibit a sufficiently high affinity interaction with collagen can promote collagen fibril network formation, stabilise clots, trap blood platelets, etc. In various embodiments, the collagen bridging molecule can be a protein or can be non-proteinaceous, such as glycosaminoglycans, or only in part proteinaceous, such as proteoglycans. In some embodiments, combinations of collagen bridging molecules can be used.

Some collagen bridging molecules are multivalent for collagen binding in their native states, such as fibronectin, which is homodimeric. Other collagen bridging molecules are monovalent collagen binders in their native states, but can be converted to multivalent ligands if they are, by way of non-limiting examples, polymerized, covalently linked together, or altered in a recombinant protein form. Examples of collagen bridging molecules are described in DiLullo et al. (2002, J. Biol, Chem., 277:4223-4231) and Sweeney et al. (2008, J. Biol. Chem, 283:21187-21197), including glycosaminoglycans (e.g., heparin and chondroitin sulfates), fibronectin, cartilage oligomeric matrix protein (COMP), secreted protein acidic and rich in aspartic acid (SPARC), various integrin receptors (e.g., α1β1 and α2β1 heterodimers and their I-domains), matrix metalloproteinase-1 (MMP-1), proteoglycans (e.g., decorin), phosphosphoryn, the platelet glycoprotein VI (GPVI) receptor, and Endol80.

In various embodiments of the kits in accordance with the present invention, the first container comprising collagen may further comprise a collagen bridging molecule in order to help torn a more cohesive intermolecular scaffold between collagen molecules.

Biological Agents

A biological agent can be incorporated into the compositions of the invention. In some embodiments, the biological agent is mixed into a solution or suspension comprising the crosslinkable collagen. In such embodiments, the biological agent will be physically incorporated into the crosslinked collagen composition upon application. In other embodiments, the biological agent is incorporated by covalent or ionic attachment. In still further embodiments, the biological agent is incorporated in the form of a microsphere. Other agents may include agents having hemostatic activity.

Biological agents may be any of several classes of compound. Where the biological agents are proteins, peptides, or polypeptides, they may be derived from natural materials, or be materials produced by recombinant DNA technology, or mutant or engineered forms of natural proteins, peptides, or polypeptides, or produced by chemical modification of proteins, peptides, or polypeptides. The classes of biostatic agents listed herein, and the particular exemplars of each class, are to be considered as exemplary rather than limiting. Biological agents may, for example, be members of the natural coagulation pathway (“coagulation factors”). Such proteins include, by way of non-limiting examples, tissue factors, factors VII, VIII, IX, and XIII, fibrin, and fibrinogen.

A biological agent may also be a protein or other compound that activates or catalyzes the natural pathways of clotting (“coagulation activators”). These include, for example, thrombin, thromboplastin, calcium (e.g. calcium glucuronate), bismuth compounds (e.g. bismuth subgallate), collagen, desmopressin and analogs, denatured collagen (gelatin), and fibronectin. Vitamin K may contribute to activation of coagulation.

Further, a biological agent may be a particulate from the class of bioactive glasses such as 45S5 glass, Combeite glass-ceramic (Na₂O—CaO-P₂O₅—SiO₂), borate bioactive glass or combinations thereof. These fillers may possess a variety of morphologies such as, but not limited to, needles, particulate, flakes, cylinders, long fibers, whiskers, or spherical particles. In preferred embodiments, the filler is comprised of particles with an average particle size ranging from less than about 1.0 μm up to a range of from 2 to 3 millimeters (mm). Preferably, the average particle size distribution ranges from about 1 to about 100 μm. The particles may be of a single size within the above noted range or may be bimodal (of two different particle sizes within the range), trimodal, etc.

A biological agent may act by local vasoconstriction (“vasoconstrictors”), such as, by way of non-limiting examples, epinephrine (adrenaline), adrenochrome, tetrahydrozoline, antihistamines (including antazoline), oxymetazoline, vasopressin and analogs thereof, and cocaine.

A biological agent may act by preventing destruction or inactivation of clotting reactions (“fibrinolysis inhibitors”), including, by way of non-limiting examples, eosinophil major basic protein, aminocaproic acid, tranexamic acid, aprotinin (Trasylol™), plasminogen activator inhibitor, plasmin inhibitor, alpha-2-macroglobulin, and adrenoreceptor blockers.

Thrombin acts as a catalyst for fibrinogen to provide fibrin, an insoluble polymer. In some embodiments, the composition of the present invention does not contain thrombin. In some embodiments, thrombin is present in the composition in an amount sufficient to catalyze polymerization of fibrinogen present in a patient's plasma. Thrombin also activates FXIII, a plasma protein that catalyzes covalent crosslinks in fibrin, rendering the resultant clot insoluble.

The fibrinogen, thrombin, FXIII or other natural protein used in the composition may be substituted by other naturally occurring or synthetic compounds or compositions which fulfill the same functions, e.g. a reptilase coagulation catalyzed, for example, ancrod, in place of thrombin.

In some embodiments, the composition of the invention will additionally comprise an effective amount of an antifibrinolytic agent to enhance the integrity of the clot as the healing process occurs. A number of antifibrinolytic agents are well known and include aprotinin, C1-esterase inhibitor and ε-amino-n-caproic acid (EACA). EACA is effective at a concentration of from about 5 mg/ml to about 40 mg/ml of the final adhesive composition, more usually from about 20 to about 30 mg/ml. EACA is commercially available as a solution having a concentration of about 250 mg/ml. Conveniently, the commercial solution is diluted with distilled water to provide a solution of the desired concentration.

Other biological factors of interest include EGF, TGF-α, TGF-β, TGF-I and TGF-II, FGF, PDGF, IFN-α, IFN-β, IL-2, IL-3, IL-6, hematopoietic factor, immunoglobulins, insulin, corticosteroids, hormones.

In some embodiments, the composition contains at least one antibiotic. The therapeutic dose levels of a wide variety of antibiotics for use in drug release systems are well known. See for example, Collagen, 1988, Vol. III, Biotechnology; Nimni, (Ed.), CRC Press, Inc., pp. 209-221 and Biomaterials, 1980, Winter et al., (Eds.), John Wiley & Sons, New York, pp. 669-676, which are incorporated herein by reference in their entirety. Anti-microbial agents are particularly useful for compositions applied to exposed wound repair sites such as sites in the mouth or to compromised wound sites such as burns.

A biological agent may comprise non-protein polymers that act to viscosity or gel, by interaction with proteins, by tamponnade, or by other mechanisms. Examples include oxidized cellulose, “Vicryl” and other polyhydroxyacids, chitosan, alginate, polyacrylic acids, pentosan polysulfate, carrageenan, and polyorthoesters (e.g., Alzamer).

A biological agent may be a material that forms a barrier to leakage by mechanical means not directly related to the natural clotting mechanisms (“barrier formers”). These include oxidized cellulose, ionically or hydrogen-bond crosslinked natural and synthetic polymers including chitin, chitosan, alginate, pectin, carboxymethylcellulose, and poloxamers, such as Pluronic surfactants.

In various embodiments of the kits in accordance with the present invention, the biological agent may be included as an additional component in either or both of the first container comprising the collagen solution and the second containing comprising the alginate solution.

### EXAMPLES

5-10 ml of a 20 mg/ml alginate solution was mixed with an approximately 20 mg/ml microfibrillar collagen solution containing 40 mM calcium chloride and 150 mM sodium chloride in a one to one ratio. The mixture was vortexed and a stiff gel was formed immediately.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A composition comprising collagen, calcium ions, and alginate, wherein the composition polymerizes in situ when contacted with blood.

2. The composition of claim 1, wherein the composition does not contain thrombin.

3. The composition of claim 1, further comprising an erythrocyte aggregation promoter.

4. The composition of claim 1, further comprising a platelet activation agent.

5. The composition of claim 3, wherein the erythrocyte aggregation promoter is a polymer having molecular weight of greater than about 20 kD.

6. The composition of claim 3, wherein the erythrocyte aggregation promoter is selected from the group consisting of poly-L-lysine, poly-L-arginine, poly-L-ornithine, protamine, poly-L-glutamine, poly-L-aspartic acid, polyethylene glycol, dextran, and mixtures thereof.

7. The composition of claim 4, wherein the platelet activation agent is selected from the group consisting of adenosine diphosphate, 1-0-alkyl-2-acetyl-sn-glyceryl-3-phosphorylcholine, arachidonic acid, epinephrine, ristocetin, cycloheximide, N-monomethyl L-arginine, atrial naturetic factor (ANF), cAMP, cGMP, ADP, prostaglandins, thromboxanes and analogs thereof, phorbols and phorbol esters, ethamsylate, hemoglobin, talc, and mixtures thereof.

8. The composition of claim 4, further comprising a collagen bridging protein selected from the group consisting of dimeric collagen-binding protein, fibronectin, glycosaminoglycans, heparin, chondroitin sulfates, cartilage oligomeric matrix protein (COMP), secreted, protein acidic and rich in aspartic acid (SPARC), integrin receptor, matrix metalloproteinase-1 (MMP-1), proteoglycan), phosphosphoryn, the platelet glycoprotein VI (GPVI) receptor, Endol80, and mixtures thereof.

9. The composition of claim 1, wherein the collagen is biotinylated.

10. The composition of claim 1, wherein the collagen is crosslinked.

11. The composition of claim 1, wherein the collagen is microfibrillar type I collagen.

12. The composition of claim 1, wherein the alginate is selected from the group consisting of sodium alginate, potassium alginate and mixtures thereof.

13. The composition of claim 1, wherein the source of the calcium ions is selected from the group consisting of calcium chloride, calcium lactate, calcium acetate and mixtures thereof.

14. The composition of claim 1, wherein the concentration of the collagen in the composition is from about 0.5 mg/ml to about 15 mg/ml.

15. The composition of claim 1, wherein the concentration of the alginate in the composition is from about 0.05 mg/ml to about 25 mg/ml.

16. The composition of claim 1, further comprising a biological agent.

17. A kit comprising a first container comprising collagen and calcium ions and a second container comprising alginate.

18. The kit of claim 17, wherein the first container and second container do not contain thrombin.

19. The kit of claim 17, wherein the concentration of the collagen is from about 1 mg/ml to about 30 mg/ml.

20. The kit of claim 17, wherein the concentration of the calcium ions is at least about 15 mM.

21. The kit of claim 17, wherein the concentration of the alginate is from about 5 mg/ml to about 20 mg/ml.

22. The kit of claim 17, wherein the first container or the second container further comprises at least one selected from the group consisting of an erythrocyte aggregation promoter, a platelet activation promoter, a collagen bridging protein, a crosslinking agent, and mixtures thereof.

23. A method of making a hemostat composition comprising:
mixing collagen, calcium ions and alginate to form a mixture; and
delivering the mixture to a site.

24. The method of claim 23, further comprising mixing the mixture comprising collagen, calcium ions and alginate with blood prior to delivering to a site of application.
